(19) United States
(12) Patent Application Publication
(10) Pub. No.: US 2025/0320231 A1
(43) Pub. Date: Oct. 16, 2025

(54) AMPHIPHILIC LIPID COMPOUND WHICH CONTAINS ZINC(II)-DIPICOLYLAMINE COMPLEX FOR NUCLEIC ACID BINDING AND LIPID NANOPARTICLE COMPOSITION COMPRISING THE SAME

(71) Applicant: KOREA INSTITUTE OF SCIENCE AND TECHNOLOGY, Seoul (KR)

(72) Inventors: Eun-Kyoung Bang, Seoul (KR); Gyochang Keum, Seoul (KR); Ansoo Lee, Seoul (KR); Soyeon Yoo, Seoul (KR)

(73) Assignee: KOREA INSTITUTE OF SCIENCE AND TECHNOLOGY, Seoul (KR)

(21) Appl. No.: 19/173,765

(22) Filed: Apr. 8, 2025

(30) Foreign Application Priority Data
Apr. 11, 2024 (KR) 10-2024-0048598

(51) Int. Cl. C07F 3/06; A61K 31/7105; A61K 47/54; A61K 48/00

(52) U.S. Cl. CPC C07F 3/06; A61K 31/7105; A61K 47/547; A61K 48/0033

(57) ABSTRACT

The present disclosure relates to an amphiphilic lipid compound which contains Zn(II)-dipicolylamine complex (Zn/DPA) for nucleic acid binding and lipid nanoparticle composition comprising the same, more specifically, the present disclosure provides an amphiphilic lipid compound for nucleic acid binding including a water-soluble linker and a hydrophobic terminal group in Zn/DPA, and a lipid nanoparticle composition including the same. By using the amphiphilic lipid compound for nucleic acid binding, it is possible to improve solubility in water and deliver nucleic acid drugs more effectively using lipid nanoparticles including the same.

### CROSS-REFERENCE TO RELATED APPLICATION

This application claims the benefit of Korean Patent Application No. 10-2024-0048598 filed on Apr. 11, 2024, in the Korean Intellectual Property Office, the entire disclosure of which is incorporated herein by reference for all purposes.

### BACKGROUND OF THE INVENTION

The present disclosure relates to an amphiphilic lipid compound which contains Zn(II)-dipicolylamine complex (Zn/DPA) for nucleic acid binding and lipid nanoparticle composition for nucleic acid delivery comprising the same.

Nucleic acid-based drugs, which began more than 40 years ago by injecting plasmid DNA into the human body to aid production of deficient proteins, have since been reported in various types such as antigens, decoys, antisense, siRNAs, and miRNAs that suppress the transcription and translation of genes. The nucleic acid-based drug has been attracting attention as a personalized therapeutic agent through complementary binding with DNA or RNA with a specific sequence by targeting DNA or RNA rather than proteins. The nucleic acid-based drug is utilized not only as a therapeutic agent but also as a prophylactic agent that defends against diseases through injection of genes capable of expressing antigens against specific diseases. While gene-based vaccines are divided into DNA vaccines, RNA vaccines, and viral vector vaccines, the RNA vaccine thereamong takes effects by injecting mRNA coding an antigen into the human body to express antigens and induce formation of antibodies against the antigen in vivo, and since there is no potential risk of infection by viral vector-based vaccines or genetic mutations by DNA vaccines, with an advantage of quick development, it has been in the spotlight as an effective measure for COVID-19 that broke out in 2019.

However, the nucleic acid-based drug is easily degraded by nucleases in the human body with poor delivery into cells as it is a negatively charged macromolecule, such that there is a need for a method to deliver the drug to a desired spot in a stable and efficient way. While delivery techniques based on various materials such as lipids, polymers, dendrimers, and inorganic metallic materials have been reported as nucleic acid delivery systems, lipid nanoparticles have been used in patisiran (ONPATTRO®), an siRNA drug first approved by FDA in 2018, as well as the mRNA vaccine for COVID-19 that was approved for emergency use in 2020. Current lipid nanoparticles are generally used in a form mixed with four components such as an ionized lipid, phospholipid (a helper lipid), cholesterol (a structure-maintaining lipid), and a PEGylated lipid in a certain ratio.

With the commercialization of new siRNA drugs and mRNA vaccines, there are a variety of issues on the lipid nanoparticle-based delivery that need to be addressed, such as targeting, easiness in storage and distribution, mitigation of side effects, cost reduction, and responses to breakthrough infections, in addition to improvement in stability and cell permeability, which are challenges in the field of traditional nucleic acid drug delivery. Therefore, the development of lipid-based deliveries and stabilizers that can solve new challenges is required in the nucleic acid drug market.

### SUMMARY OF THE INVENTION

An object of the present disclosure is to provide an amphiphilic lipid which is based on Zn(II)-dipicolylamine complex (Zn/DPA) has a water-soluble linker and a hydrophobic terminal, and a lipid nanoparticle composition for nucleic acid delivery comprising the same.

To achieve the above object, the present disclosure provides a compound selected from a compound represented by the following Chemical Formula 1 or a pharmaceutically acceptable salt thereof:

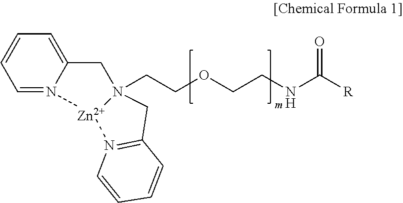

wherein, in the Chemical Formula 1, R is a linear or branched, saturated or unsaturated hydrocarbon with 6 to 22 carbon atoms, where the hydrocarbon includes or does not include an ester, ether, amide, carbamate, carbonate, or disulfide bond, and the ester, ether, amide, carbamate, carbonate, or disulfide bond is bonded or not bonded to a glycerol structure, and m is an integer of 1 to 10.

The present disclosure provides a compound selected from a compound represented by the following Chemical Formula 7 or a pharmaceutically acceptable salt thereof:

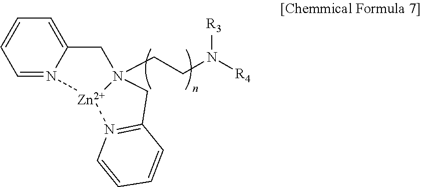

wherein, in the Chemical Formula 7, R₃ and R₄ are the same or different and each independently linear or branched, saturated or unsaturated hydrocarbons with 6 to 22 carbon atoms, where the hydrocarbon includes or does not include an ester, ether, amide, carbamate, carbonate, or disulfide bond, and the ester, ether, amide, carbamate, carbonate, or disulfide bond is bonded or not bonded to a glycerol structure, and n is an integer of 0 to 10.

The present disclosure provides a compound selected from a compound represented by the following Chemical Formula 11 or a pharmaceutically acceptable salt thereof:

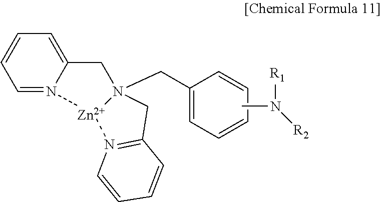

wherein, in the Chemical Formula 11, R₁ and R₂ are the same or different and each independently linear or branched, saturated or unsaturated hydrocarbons with 6 to 22 carbon atoms, where the hydrocarbon includes or does not include an ester, ether, amide, carbamate, carbonate, or disulfide bond, and the ester, ether, amide, carbamate, carbonate, or disulfide bond is bonded or not bonded to a glycerol structure.

The present disclosure provides an amphiphilic lipid compound for nucleic acid binding, including the compound.

The present disclosure provides a lipid nanoparticle composition including the amphiphilic lipid compound for nucleic acid binding.

In addition, the present disclosure provides a composition for drug delivery, including the lipid nanoparticle composition; and a therapeutic or prophylactic agent.

A novel compound according to the present disclosure includes a water-soluble linker and a hydrophobic terminal group in Zn/DPA, thereby improving solubility in water.

By utilizing the compound as an amphiphilic lipid compound for nucleic acid binding, it is possible to prepare lipid nanoparticles and deliver nucleic acid drugs more effectively using the same.

### BRIEF DESCRIPTION OF THE DRAWINGS

FIG. 1 shows a result of analyzing changes in fluorescence intensity of dihydroxy-4-sulformethyl coumarin according to RNA concentration for amphiphilic lipid compounds (Compounds with Chemical Formulas 3 to 6).

FIG. 2 shows a result of analyzing changes in fluorescence intensity of dihydroxy-4-sulformethyl coumarin according to RNA concentration for amphiphilic lipid compounds (Compounds with Chemical Formulas 9, 10, 13, and 14).

FIG. 3 shows results of analyzing gel chromatography of an amphiphilic lipid compound-nucleic acid complex according to Zn/P, which is a ratio of phosphate residues in RNA to Zn of amphiphilic lipid compounds (Compounds with Chemical Formulas 3 to 6).

FIG. 4 shows results of analyzing gel chromatography of an amphiphilic lipid compound-nucleic acid complex according to Zn/P, which is a ratio of phosphate residues in RNA to Zn of amphiphilic lipid compounds (Compounds with Chemical Formulas 9, 10, 13, and 14).

### DETAILED DESCRIPTION

Hereinafter, the present disclosure will be described in detail.

The present inventor completed the present disclosure by synthesizing a novel amphiphilic lipid based on Zn(II)-dipicolylamine complex (Zn/DPA) in order to improve shortcomings due to conventional lipid nanoparticles and finding the probability in production of lipid nanoparticles that are capable of appropriately delivering nucleic acid drugs using the same.

The present disclosure provides an amphiphilic lipid compound based on Zn/DPA.

More specifically, the present disclosure provides a compound selected from a compound represented by the following Chemical Formula 1 or a pharmaceutically acceptable salt thereof:

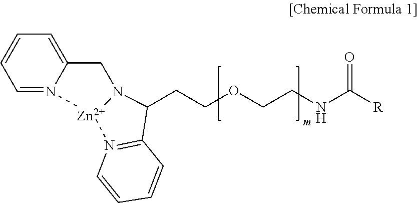

wherein, in the Chemical Formula 1, R may be a linear or branched, saturated or unsaturated hydrocarbon with 6 to 22 carbon atoms, where the hydrocarbon may or may not include an ester, ether, amide, carbamate, carbonate, or disulfide bond, and the ester, ether, amide, carbamate, carbonate, or disulfide bond may or may not be bonded to a glycerol structure, and m may be selected from integers of 1 to 10.

Preferably, the compound represented by Chemical Formula 1 may be a compound represented by the following Chemical Formula 2:

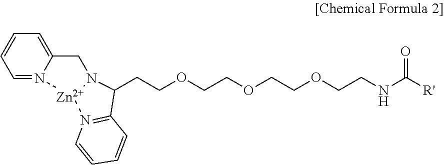

wherein, in the Chemical Formula 2, R′ may be a linear or branched, saturated or unsaturated hydrocarbon with 6 to 22 carbon atoms, where the hydrocarbon may or may not include an ester, ether, amide, carbamate, carbonate, or disulfide bond.

More preferably, the compound represented by Chemical Formula 1 may include any one or more selected from the group consisting of compounds represented by the following Chemical Formula 3 to Chemical Formula 6:

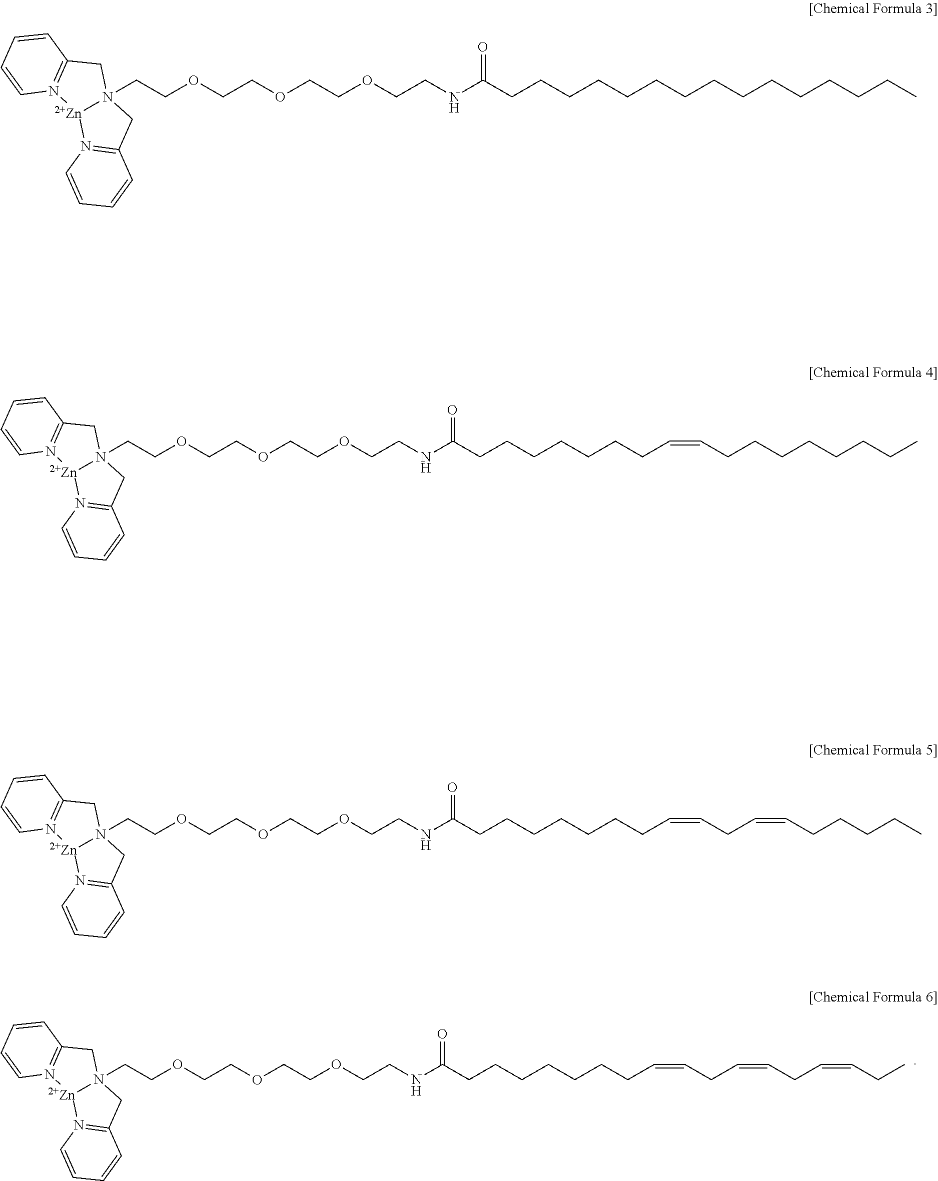

As used herein, the compound represented by Chemical Formula 3 may be named Zn/DPA-PA, the compound represented by Chemical Formula 4 named Zn/DPA-OA, the compound represented by Chemical Formula 5 named Zn/DPA-LA, and the compound represented by Chemical Formula 6 named Zn/DPA-LNA.

The compound represented by the following Chemical Formula 1 according to the present disclosure includes a water-soluble linker and a hydrophobic terminal group in Zn/DPA, thereby improving solubility in water.

The present disclosure provides a compound selected from a compound represented by the following Chemical Formula 7 or a pharmaceutically acceptable salt thereof:

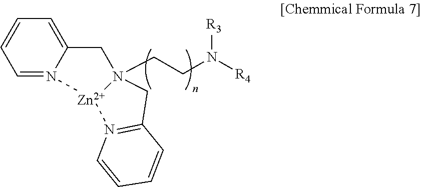

wherein, in the Chemical Formula 7, R₃ and R₄ may be the same or different and each independently linear or branched, saturated or unsaturated hydrocarbons with 6 to 22 carbon atoms, where the hydrocarbon may or may not include an ester, ether, amide, carbamate, carbonate, or disulfide bond, and the ester, ether, amide, carbamate, carbonate, or disulfide bond may or may not be bonded to a glycerol structure, and n may be selected from integers of 0 to 10.

Preferably, the compound represented by Chemical Formula 7 may be a compound represented by the following Chemical Formula 8:

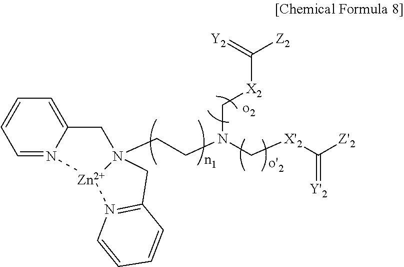

wherein, in the Chemical Formula 8, X₂ and X′₂ may be the same or different and each independently CH₂, NH, or O; Y₂ and Y′₂ may be the same or different and each independently O or S; Z₂ and Z′₂ may be the same or different and each independently linear or branched, saturated or unsaturated hydrocarbons with 6 to 22 carbon atoms; n₁ may be an integer of 0 to 5; and o₂ and o′₂ may be the same or different and each independently selected from integers of 2 to 10.

Specifically, in the Chemical Formula 8, X₂ and X′₂ may be the same or different and each independently CH₂, NH, or O; Y₂ and Y′₂ may be O; Z₂ and Z′₂ may be the same or different and each independently linear or branched, saturated or unsaturated hydrocarbons with 6 to 22 carbon atoms; n₁ may be an integer of 0 to 3; and o₂ and o′₂ may be the same or different and each independently integers of 4 to 8.

More preferably, the compound represented by Chemical Formula 7 may include a compound represented by the following Chemical Formula 9 or Chemical Formula 10:

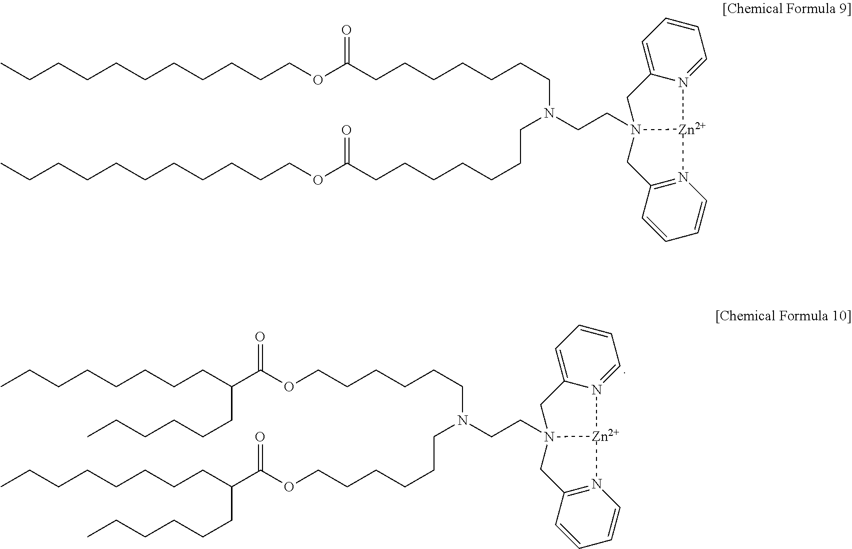

As used herein, the compound represented by Chemical Formula 9 may be named Zn/DPA-01 and the compound represented by Chemical Formula 10 may be named Zn/DPA-02.

The compound represented by Chemical Formula 7 according to the present disclosure may include a terminal group exhibiting hydrophobicity in Zn/DPA, and the terminal group may have a cone shape to help the endosomal escape of nucleic acids.

In addition, the present disclosure provides a compound selected from a compound represented by the following Chemical Formula 11 or a pharmaceutically acceptable salt thereof:

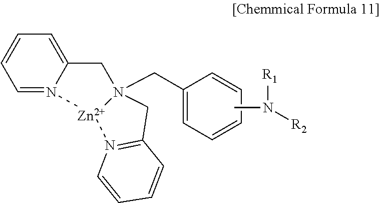

wherein, in the Chemical Formula 11, R₁ and R₂ may be the same or different and each independently linear or branched, saturated or unsaturated hydrocarbons with 6 to 22 carbon atoms, where the hydrocarbon may or may not include an ester, ether, amide, carbamate, carbonate, or disulfide bond, and the ester, ether, amide, carbamate, carbonate, or disulfide bond may or may not be bonded to a glycerol structure.

Preferably, the compound represented by Chemical Formula 11 may be a compound represented by the following Chemical Formula 12:

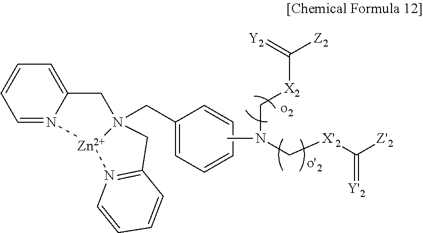

wherein, in the Chemical Formula 12, X₁ and X′₁ may be the same or different and each independently CH₂, NH, or O; Y₁ and Y₁ may be the same or different and each independently O or S; Z₁ and Z′1 may be the same or different and each independently linear or branched, saturated or unsaturated hydrocarbons with 6 to 22 carbon atoms; and 01 and o′1 may be the same or different and each independently selected from integers of 2 to 10.

Specifically, in the Chemical Formula 12, X₁ and X′₁ may be the same or different and each independently CH₂, NH, or O; Y₁ and Y₁ may be O; Z₁ and Z′₁ may be the same or different and each independently linear or branched, saturated or unsaturated hydrocarbons with 6 to 22 carbon atoms; o₁ and o′₁ may be the same or different and each independently integers of 4 to 8; and nitrogen bonded to benzene may be bound to the ortho-, meta-, or para-position.

More preferably, the compound represented by Chemical Formula 11 may include a compound represented by the following Chemical Formula 13 or Chemical Formula 14:

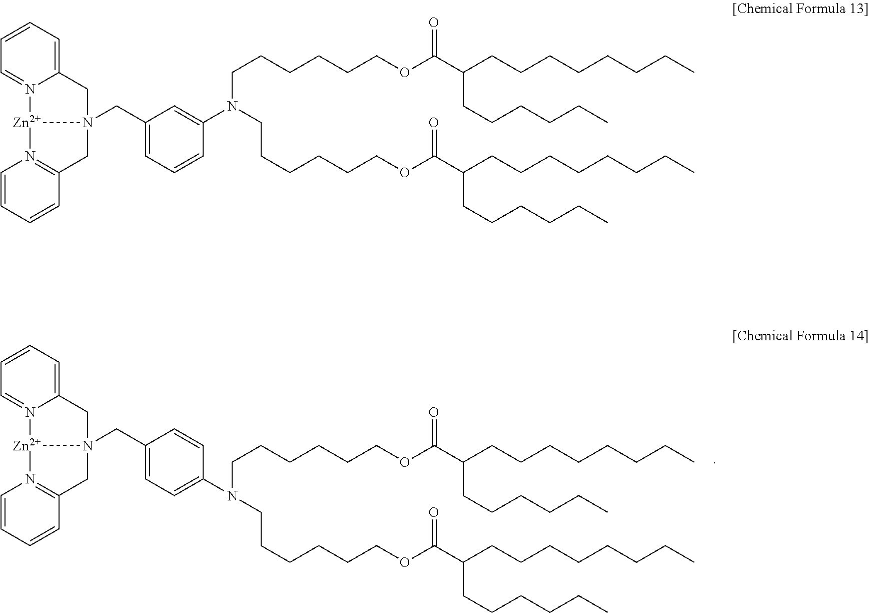

As used herein, the compound represented by Chemical Formula 13 may be named Zn/DPA-03, and the compound represented by Chemical Formula 14 may be named Zn/DPA-04.

The compound may include a form of pharmaceutically acceptable salts that exhibit the same or similar activity.

As used herein, the term “pharmaceutically acceptable” refers to a state of securing safety and efficacy profiles suitable for administration to humans since there is no toxicity to cells or humans exposed to the compound or salt thereof.

The salt may be used in any one form of a basic salt or an acidic salt that is pharmaceutically or foodologically acceptable. The basic salt may be used in any one form of organic or inorganic base salts and selected from the group consisting of sodium salts, potassium salts, calcium salts, lithium salts, magnesium salts, cesium salts, aminium salt, ammonium salts, triethylamine salts, and pyridinium salts.

Acid addition salt that is formed by free acid is useful as the acidic salt. Inorganic acids and organic acids may be used as the free acids, hydrochloric acid, bromic acid, sulfuric acid, sulfurous acid, phosphoric acid, diphosphoric acid, and nitric acid may be used as the inorganic acid, whereas citric acid, acetic acid, maleic acid, malic acid, fumaric acid, gluconic acid, methanesulfonic acid, benzenesulfonic acid, camphorsulfonic acid, oxalic acid, malonic acid, glutaric acid, acetic acid, glycolic acid, succinic acid, tartaric acid, 4-toluenesulfonic acid, galacturonic acid, embonic acid, glutamic acid, citric acid, aspartic acid, and stearic acid may be used as the organic acid, and salts formed using various inorganic acids and organic acids that are commonly used in the art may all be included without limitation.

In addition, the compound may include not only the salt described above, but also all salts, hydrates, solvates, and derivatives, which may be prepared by conventional methods. The addition salt may be prepared by conventional methods, for example, by dissolving the compound in a water-miscible organic solvent, e.g., acetone, methanol, ethanol, or acetonitrile, and then carrying precipitation or crystallization after adding an excess of organic bases or an aqueous basic solution of inorganic bases. Alternatively, preparation may be carried out by obtaining the addition salt by evaporation of the solvent or excess base from the mixture followed by drying or suction filtration of the precipitated salts.

The present disclosure provides an amphiphilic lipid compound for nucleic acid binding, including the compound.

Corresponding features may be substituted in the above-mentioned sections.

The present disclosure provides a lipid nanoparticle composition including an amphiphilic lipid compound for nucleic acid binding.

The composition may further include one or more selected from the group consisting of a helper lipid and a PEGylated lipid.

The helper lipid may be any one or more selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinoleoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinoleoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl-phosphatidyl-ethanolamine (DPPE), 1,2-dimyristoyl-sn-glycero-3-phosphoethanolamine (DMPE), 1-stearoyl-2-oleoyl-phosphatidylethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), alkyl gallate, and trehalose derivatives, and the alkyl gallate may be selected from propyl gallate, dodecyl gallate, hexadecyl gallate, octadecyl gallate (or stearyl gallate), or cis-9-octadecenyl gallate, preferably, octadecyl gallate or trehalose dioleate, but is not limited thereto.

The polyethylene glycol (PEG)-modified lipid may be selected from the group consisting of PEG-modified phosphatidylethanolamine, PEG-modified phosphatidic acid, PEG-modified ceramide, PEG-modified dialkylamine, PEG-modified diacylglycerol, PEG-modified dialkyl glycerol, and a mixture thereof, preferably myristoyl diglyceride (DMG)-PEG, but is not limited thereto.

The composition may include 50 to 80 mol % of the amphiphilic lipid compound for nucleic acid binding, 5 to 20 mol % of the helper lipid, and 1 to 5 mol % of the PEGylated lipid, preferably it may include, but is not limited to, 60 to 80 mol % of the amphiphilic lipid compound for nucleic acid binding, 5 to 15 mol % of the helper lipid, and 1 to 5 mol % of the PEGylated lipid.

More preferably, the composition may include any one or more compounds selected from the group consisting of compounds represented by Chemical Formulas 3 to 6, 9, 10, 13, and 14 as amphiphilic lipid compounds for nucleic acid binding, stearyl gallate or trehalose dioleate as the helper lipid, and myristoyl diglyceride (DMG-PEG) as the PEGylated lipid.

The composition may additionally include a therapeutic or prophylactic agent.

The composition may be formed solely with any one compound selected from the group consisting of compounds represented by Chemical Formulas 3 to 6, 9, 10, 13, and 14, or in combination with the therapeutic or prophylactic agent.

The therapeutic or prophylactic agent may be a vaccine or compound capable of inducing an immune response, or may be selected from the group consisting of small interfering RNA (siRNA), asymmetric interfering RNA (aiRNA), microRNA (miRNA), dicer-matrix RNA (dsRNA), small hairpin RNA (shRNA), messenger RNA (mRNA), and a mixture thereof, but is not limited thereto.

The encapsulation efficiency of the therapeutic or prophylactic agent may independently be at least 50% or greater, and a WT/WT ratio of the therapeutic or prophylactic agent to the lipid component may be about 10:1 to about 60:1.

The N:P ratio of the therapeutic or prophylactic agent may be from about 2:1 to about 30:1. The N/P ratio is a value obtained by dividing the number of ionizable nitrogen in ionized lipids by the number of phosphate groups in nucleic acid molecules.

The lipid nanoparticle composition may be used for nucleic acid delivery.

The present disclosure provides a composition for drug delivery, including the lipid nanoparticle composition; and a therapeutic or prophylactic agent.

Corresponding features may be substituted in the above-mentioned sections.

The present disclosure provides a method of delivering a therapeutic or prophylactic agent to mammalian cells by the composition for drug delivery.

The method of delivering the therapeutic or prophylactic agent to the mammalian cell may include administering the lipid nanoparticle composition to a subject, but the dosing is conducted by bringing the cell in contact with the nanoparticle composition to allow the therapeutic or prophylactic agent to be delivered to the cell.

The mammalian cells belong to the mammal.

The mammal may be human.

In addition, the composition for drug delivery may be administered intravenously, intramuscularly, intradermally, subcutaneously, intranasally, or by inhalation. Doses of the therapeutic and/or prophylactic agent ranging from about 0.01 mg/kg to about 10 mg/kg may be administered to mammals.

In addition, the present disclosure provides an immunoenhancing composition including the lipid nanoparticle composition.

Corresponding features may be substituted in the above-mentioned sections.

Hereinafter, to help the understanding of the present disclosure, examples will be described in detail. However, the following examples are merely illustrative of the content of the present disclosure, and the scope of the present disclosure is not limited to the following examples. The examples of the present disclosure are provided to more completely explain the present disclosure to those skilled in the art.

### <Example 1> Synthesis of Amphiphilic Lipids

### 1. Synthesis of Zn/DPA-PA, Zn/DPA-OA, Zn/DPA-LA, and Zn/DPA-LNA

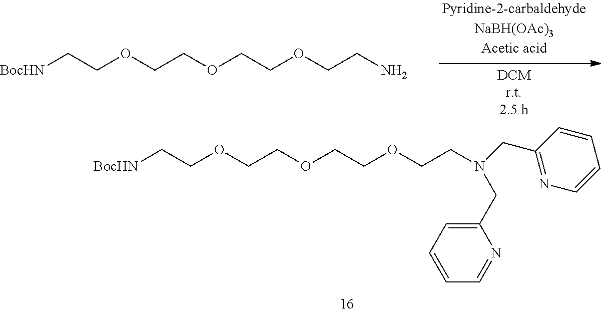 [SMILES: CC(C)(C)OC(=O)NCCOCCOCCOCCN ; CC(C)(C)OC(=O)NCCOCCOCCOCCN(Cc1ccccn1)Cc1ccccn1]

13-Amino-5,8,11-trioxa-2-azatridecanoic acid 1,1-dimethylethyl ester (1.2 g, 4.1 mmol) was dissolved in DCM (25 mL), and then pyridine-2-carbaldehyde (3 equiv.) and acetic acid (3 equiv.) were added, followed by stirring at room temperature for 30 minutes. Thereafter, NaBH(OAc)₃ (3 equiv.) was additionally added and stirred at room temperature for 2 hours. Once the reaction was completed, DCM (75 mL) was additionally added followed by washing with 1N aqueous NaOH solution, and a filtrate filtered by removing moisture from the obtained organic layer with anhydrous Na₂SO₄ was distilled under reduced pressure and dried under vacuum. Compound 16, obtained in yellow oil, was used in the next step without any further purification process.

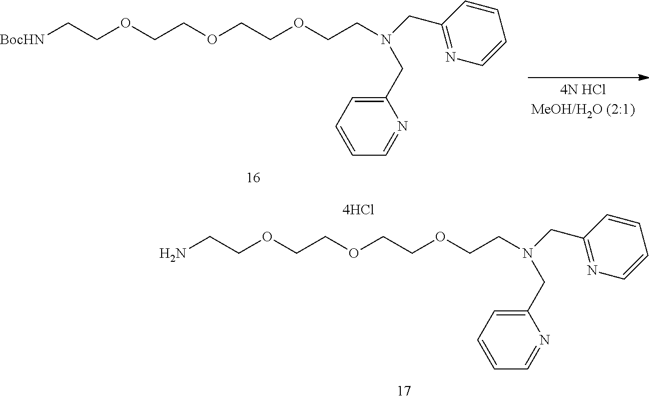 [SMILES: CC(C)(C)OC(=O)NCCOCCOCCOCCN(Cc1ccccn1)Cc1ccccn1 ; NCCOCCOCCOCCN(Cc1ccccn1)Cc1ccccn1]

35-37% aqueous HCl solution and methanol were mixed in 1:2, and 45 mL of prepared 4N HCl solution was added to dry Compound 16 to dissolve at a concentration of 0.1 M, followed by stirring for 5 hours. Once the reaction was terminated, the mixture was distilled under reduced pressure with the further addition of MeOH and EtOAc until a solid was formed with HCl salts. After dissolving the resulting reaction mixture in MeOH (5 mL), it was added to diethyl ether (40 mL) dropwise to filter out the precipitated solids and then dried under vacuum to obtain Compound 17 (2.4 g, quant.) quantitatively.

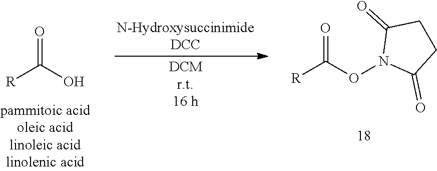

Fatty acids (palmitoic acid, oleic acid, linoleic acid, linolenic acid) were dissolved in DCM at a level of 0.3 M, and N-hydroxysuccinimide (1.3 equiv.) and dicyclohexylcarbodiimide (1.3 equiv.) were added, followed by stirring at room temperature for 6 hours. After checking that the reaction was terminated via TLC, a filtrate obtained by filtering the resulting dicyclohexylurea was distilled under reduced pressure and purified by column chromatography (SiO₂:DCM:Hexane=1:1 to DCM only) to obtain Compound 18 (91-96%), succinyl ester of fatty acids, in white solid.

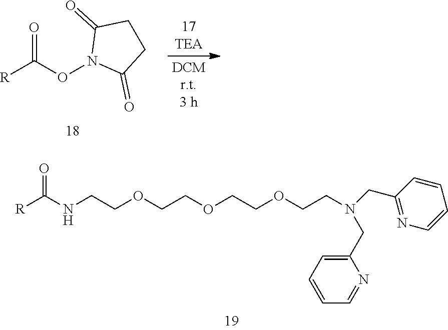

After dissolving Compound 18 in DCM at a level of 0.1 M, Compound 17 (1.1 equiv.) and triethylamine (TEA, 5.5 equiv.) were added sequentially and stirred for 3 hours. After checking that the reaction was terminated via TLC, DCM was additionally added for dilution, organic layers washed with saturated aqueous NaHCO₃ solution (×2) were collected, and then a filtrate filtered by removing moisture from the organic layer with anhydrous Na₂SO₄ was concentrated by decompression distillation. The concentrated mixture was purified by column chromatography (basic alumina: EtOAc only to 1% MeOH in EtOAc) to obtain Compound 19 (35˜62%) in white solid.

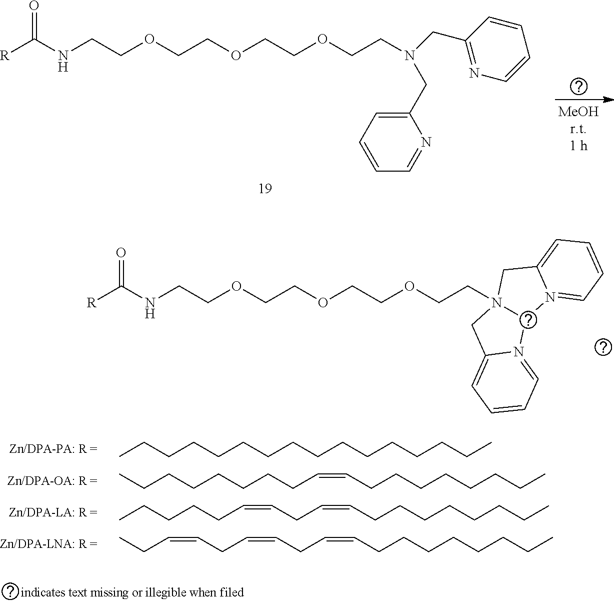

Compound 19 was dissolved in methanol at a concentration of 0.1 M, and then zinc nitrate hexahydrate (1.05 equiv.) was added, followed by stirring for an hour. After checking that the reaction was terminated via TLC, the reaction mixture solution was filtered through a cotton filter to remove impurities, followed by distillation under reduced pressure. The reaction mixture was re-dissolved in MeOH (1 mL) and added dropwise in diethyl ether (30 mL) to settle the precipitated white solid by centrifugation, and the liquid portion was discarded. The obtained solids were washed with diethyl ether (10 mL×2) and then dried under vacuum to obtain Zn/DPA-PA, Zn/DPA-OA, Zn/DPA-LA, and Zn/DPA-LNA compounds (90-99%).

Zn/DPA-PA: (Compound with Chemical Formula 3)

¹H NMR (400 MHz, MeOD): δ 8.77 (dt, J=5.2, 1.3 Hz, 2H), 8.15 (td, J=7.7, 1.6 Hz, 2H), 7.69 (ddd, J=8.1, 6.7, 1.3 Hz, 4H), 4.49 (d, J=16.3 Hz, 2H), 4.28 (d, J=16.3 Hz, 2H), 3.61-3.58 (m, 8H), 3.52 (t, J=5.8 Hz, 2H), 3.49-3.46 (m, 2H), 3.44-3.42 (m, 2H), 3.33 (t, J=5.6 Hz, 2H), 3.02 (t, J=4.8 Hz, 2H), 2.16 (t, J=7.6 Hz, 2H), 1.51-1.56 (m, 2H), 1.31-1.28 (m, 24H), 0.90 (t, J=6.8 Hz, 3H)

Zn/DPA-OA: (Compound with Chemical Formula 4)

¹H NMR (400 MHz, MeOD) δ 8.77 (dt, J=5.3, 1.3 Hz, 2H), 8.15 (td, J=7.7, 1.7 Hz, 2H), 7.79-7.60 (m, 4H), 5.38-5.30 (m, 2H), 4.48 (d, J=16.3 Hz, 2H), 4.28 (d, J=16.3 Hz, 2H), 3.62-3.59 (m, 8H), 3.52 (t, J=5.8 Hz, 2H), 3.50-3.46 (m, 2H), 3.44-3.41 (m, 2H), 3.33 (t, J=5.7 Hz, 2H), 3.02 (t, J=5.0 Hz, 2H), 2.17 (t, J=7.5 Hz, 2H), 2.05-2.00 (m, 4H), 1.61-1.54 (m, 2H), 1.40-1.23 (m, 20H), 0.90 (t, J=6.8 Hz, 3H).

Zn/DPA-LA: (Compound with Chemical Formula 5)

¹H NMR (400 MHz, MeOD) δ 8.80 (dt, J=5.2, 1.3 Hz, 2H), 8.18 (td, J=7.7, 1.7 Hz, 2H), 7.81-7.63 (m, 4H), 5.51-5.27 (m, 4H), 4.51 (d, J=16.3 Hz, 2H), 4.31 (d, J=16.3 Hz, 2H), 3.68-3.58 (m, 8H), 3.54 (t, J=5.8 Hz, 2H), 3.52-3.49 (m, 2H), 3.46-3.44 (m, 2H), 3.37 (t, J=5.7 Hz, 2H), 3.05 (t, J=5.0 Hz, 2H), 2.80 (t, J=6.3 Hz, 2H), 2.20 (t, J=7.5 Hz, 2H), 2.12-2.06 (m, 4H), 1.67-1.55 (m, 2H), 1.45-1.27 (m, 14H), 0.93 (t, J=6.8 Hz, 3H).

Zn/DPA-LNA: (Compound with Chemical Formula 6)

¹H NMR (400 MHz, MeOD): δ 8.80 (d, J=4.8 Hz, 2H), 8.18 (td, J=7.8, 1.7 Hz, 2H), 7.79-7.67 (m, 4H), 5.63-5.20 (m, 6H), 4.51 (d, J=16.3 Hz, 2H), 4.31 (d, J=16.3 Hz, 2H), 3.64-3.62 (m, 8H), 3.54 (t, J=5.8 Hz, 2H), 3.52-3.49 (m, 2H), 3.45 (t, J=5.0 Hz, 3H), 3.36 (t, J=5.7 Hz, 2H), 3.04 (t, J=4.9 Hz, 2H), 2.84 (t, J=5.9 Hz, 4H), 2.20 (t, J=7.5 Hz, 2H), 2.15-2.07 (m, 4H), 1.61 (p, J=7.2 Hz, 2H), 1.44-1.26 (m, 8H), 1.00 (t, J=7.5 Hz, 2H)

### 2. Synthesis of Zn/DPA-01, 02, 03, 04

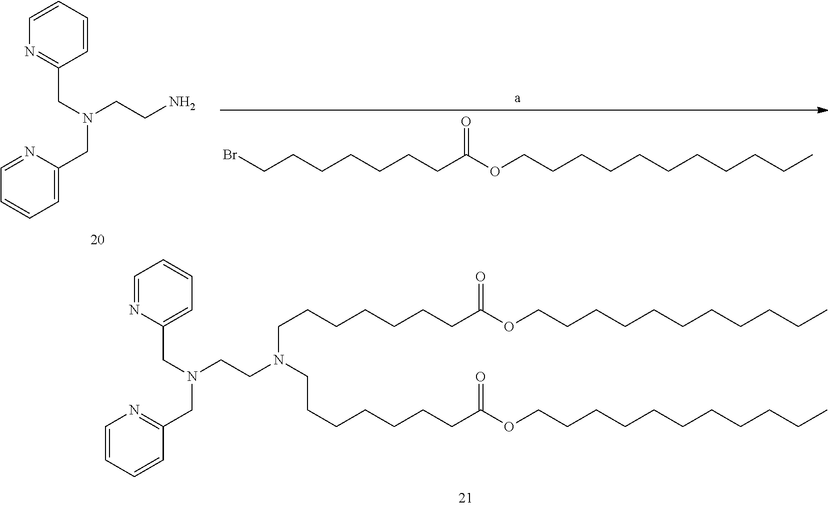 [SMILES: NCCN(Cc1ccccn1)Cc1ccccn1 ; CCCCCCCCCCCOC(=O)CCCCCCCBr ; CCCCCCCCCCCOC(=O)CCCCCCCN(CCCCCCCC(=O)OCCCCCCCCCCC)CCN(Cc1ccccn1)Cc1ccccn1]

Step a: Compound 20 (0.12 mmol), undecyl 8-bromooctanoate (0.24 mmol), K₂CO₃ (1.2 mmol), and KI (0.024 mmol) were dissolved in 2.0 mL of MeCN in the presence of nitrogen, followed by stirring at 90° C. for 24 hours. After terminating the reaction by adding water, organic matter was extracted using DCM, and excess water was removed with MgSO₄. After removing the solvent by decompression distillation, it was separated using basic alumina column chromatography (ethyl acetate:hexane=1:4) to obtain Compound 21 (50.9 mg, 50% yield) in light brown oil.

HRMS (ESI) calculated for C₅₂H₉₁N₄O₄ [M+H]⁺: 835.7040, Found: 835.7038.

¹H NMR (400 MHz, CDCl₃): δ 8.47 (dd, J=5.01, 1.47 Hz, 2H), 7.60 (td, J=7.64, 2.08 Hz, 2H), 7.50 (d, J=7.70 Hz, 2H), 7.09 (dd, J=7.70, 4.65 Hz, 2H), 4.01 (t, J=6.79 Hz, 4H), 3.81 (s, 4H), 2.71-2.43 (m, 4H), 2.28 (t, J=7.40 Hz, 3H), 2.23 (t, J=7.52 Hz, 4H), 1.56 (h, J=7.83, 7.34 Hz, 8H), 1.22 (d, J=4.52 Hz, 50H), 0.83 (t, J=6.85 Hz, 6H).

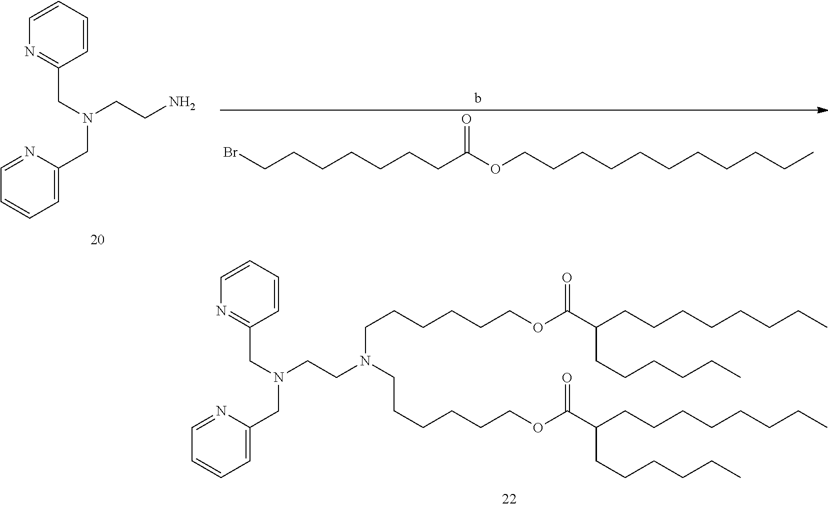 [SMILES: NCCN(Cc1ccccn1)Cc1ccccn1 ; CCCCCCCCCCCOC(=O)CCCCCCCBr ; CCCCCCCCC(CCCCCC)C(=O)OCCCCCCN(CCCCCCOC(=O)C(CCCCCC)CCCCCCCC)CCN(Cc1ccccn1)Cc1ccccn1]

Step b: Compound 20 (0.28 mmol), 6-oxohexyl 2-hexyldecanoate (0.59 mmol), acetic acid (0.52 mmol), and NaBH(OAc)₃ (1.68 mmol) were dissolved in 7.7 mL of DCM in the presence of nitrogen, followed by stirring for 1 hour at room temperature. After adding 2M aqueous NaOH solution to terminate the reaction, organic matter was extracted using DCM, and excess water was removed with MgSO₄. After removing the solvent through decompression distillation, the mixture was separated using basic alumina column chromatography (ethyl acetate:hexane=1:4) to obtain Compound 22 (84.9 mg, 33% yield) in light brown oil.

HRMS (ESI) calculated for C₅₈H₁₀₃N₄O₄ [M+H]⁺: 919.7979, Found: 919.7974.

¹H NMR (400 MHz, CDCl₃): δ 8.44 (d, J=5.26 Hz, 2H), 7.56 (t, J=7.70 Hz, 2H), 7.47 (d, J=7.82 Hz, 2H), 7.14-6.96 (m, 2H), 3.97 (t, J=6.72 Hz, 4H), 3.79 (s, 4H), 2.57 (s, 4H), 2.26 (s, 6H), 1.65-1.44 (m, 9H), 1.17 (s, 53H), 0.79 (t, J=6.79 Hz, 14H).

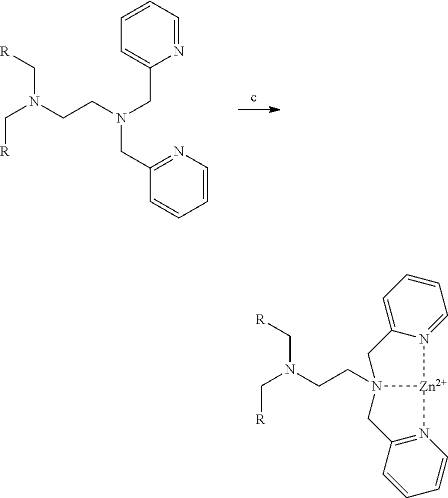

Step c: The lipid (0.1 mmol) synthesized in the presence of nitrogen and Zn²⁺ salt (0.1 mmol) were dissolved in 1.0 mL of MeCN and stirred at room temperature for 3 hours, and the solvent was removed through decompression distillation.

Zn/DPA-01: (Compound with Chemical Formula 9)

¹H NMR (400 MHz, CDCl₃): δ 9.04 (s, 2H), 7.94 (d, J=7.70 Hz, 2H), 7.72 (d, J=8.19 Hz, 2H), 7.47 (t, J=6.66 Hz, 2H), 4.85-4.25 (m, 4H), 3.96 (d, J=12.23 Hz, 4H), 3.36 (s, 10H), 3.16 (s, 2H), 2.81 (s, 2H), 2.59 (s, 4H), 2.16 (s, 4H), 1.64-1.34 (m, 12H), 1.18 (s, 35H), 0.79 (s, 6H).

Zn/DPA-02: (Compound with Chemical Formula 10)

¹H NMR (400 MHz, CDCl₃): δ 9.03 (d, J=5.62 Hz, 2H), 7.95 (t, J=7.70 Hz, 2H), 7.87 (d, J=7.95 Hz, 2H), 7.45 (t, J=6.48 Hz, 2H), 4.87-4.44 (m, 4H), 3.93 (t, J=6.79 Hz, 5H), 3.30 (t, J=5.26 Hz, 2H), 2.85 (t, J=5.14 Hz, 2H), 2.62 (t, J=8.25 Hz, 4H), 2.24 (dt, J=8.80, 3.73 Hz, 2H), 1.45 (dddd, J=37.54, 29.96, 14.24, 7.21 Hz, 20H), 1.20 (s, 39H), 0.81 (t, J=6.79 Hz, 16H).

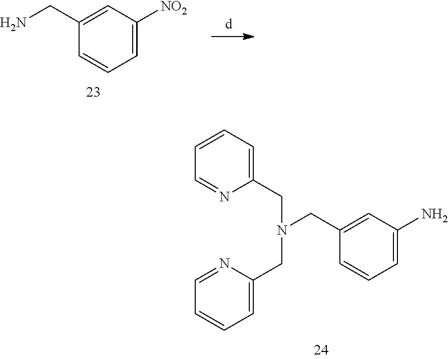 [SMILES: NCc1cccc([N+](=O)[O-])c1 ; Nc1cccc(CN(Cc2ccccn2)Cc2ccccn2)c1]

Step d: Compound 23 (0.78 mmol), picolinaldehyde (1.56 mmol), and NaBH(OAc)₃ (3.90 mmol) were dissolved in 10 mL of DCE, followed by stirring at room temperature for 1 hour. After adding 2M aqueous NaOH solution to terminate the reaction, organic matter was extracted using DCM, and excess water was removed using MgSO₄. After removing the solvent through decompression distillation, SnCl₂·2H₂O (3.70 mmol) and 20 mL of EtOH were added and stirred at 100° C. for 12 hours. After terminating the reaction by adding a saturated aqueous NaHCO₃ solution, organic matter was extracted using DCM, and excess water was removed with MgSO₄.

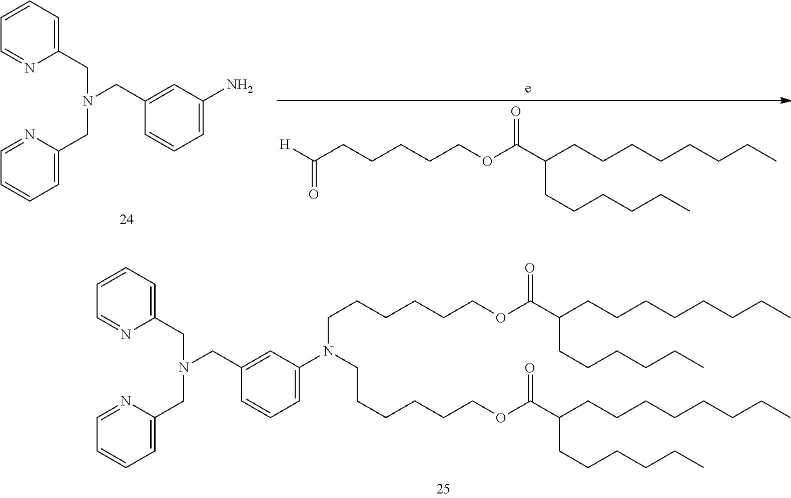 [SMILES: Nc1cccc(CN(Cc2ccccn2)Cc2ccccn2)c1 ; CCCCCCCCC(CCCCCC)C(=O)OCCCCCC=O ; CCCCCCCCC(CCCCCC)C(=O)OCCCCCCN(CCCCCCOC(=O)C(CCCCCC)CCCCCCCC)c1cccc(CN(Cc2ccccn2)Cc2ccccn2)c1]

Step e: Compound 24 (0.78 mmol), 6-oxohexyl 2-hexyldecanoate (1.56 mmol), acetic acid (1.17 mmol), and NaBH(OAc)₃ (4.51 mmol) were dissolved in 10 mL of DCM in the presence of nitrogen, followed by stirring at room temperature for 1 hour. After terminating the reaction by adding 2M aqueous NaOH solution, organic matter was extracted using DCM, and excess water was removed with MgSO₄. After removing the solvent through decompression distillation, the mixture was separated using basic alumina column chromatography (ethyl acetate:hexane=1:4) to obtain Compound 25 (283.3 mg, 37% yield) in light brown oil.

¹H NMR (400 MHz, CDCl₃): δ 8.48 (s, 2H), 7.60 (d, J=4.77 Hz, 4H), 7.14-6.98 (m, 3H), 6.74-6.63 (m, 2H), 6.48 (d, J=5.50 Hz, 1H), 4.04 (d, J=13.20 Hz, 4H), 3.81 (s, 4H), 3.62 (s, 2H), 3.44-3.12 (m, 4H), 2.29 (dt, J=9.05, 3.91 Hz, 2H), 1.59 (dq, J=15.53, 8.56, 7.83 Hz, 12H), 1.40 (dd, J=14.61, 5.69 Hz, 12H), 1.22 (s, 39H), 0.84 (t, J=6.72 Hz, 13H).

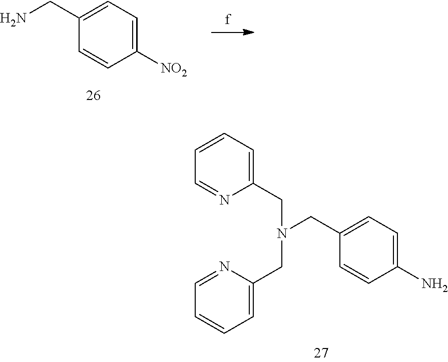 [SMILES: NCc1ccc([N+](=O)[O-])cc1 ; Nc1ccc(CN(Cc2ccccn2)Cc2ccccn2)cc1]

Step f: Compound 26 (0.95 mmol), picolinaldehyde (1.90 mmol), and NaBH(OAc)₃ (4.76 mmol) were dissolved in 12 mL of DCE, followed by stirring at room temperature for 1 hour. After terminating the reaction by adding 2M aqueous NaOH solution, organic matter was extracted using DCM, and excess water was removed with MgSO₄. After removing the solvent through decompression distillation, Fe (4 equiv.), NH₄Cl (0.5 equiv.), and 5 mL of H₂O were added and then stirred at 130° C. for 2 hours. After the reaction was completed, the organic matter was extracted using DCM, and the excess water was removed with MgSO₄.

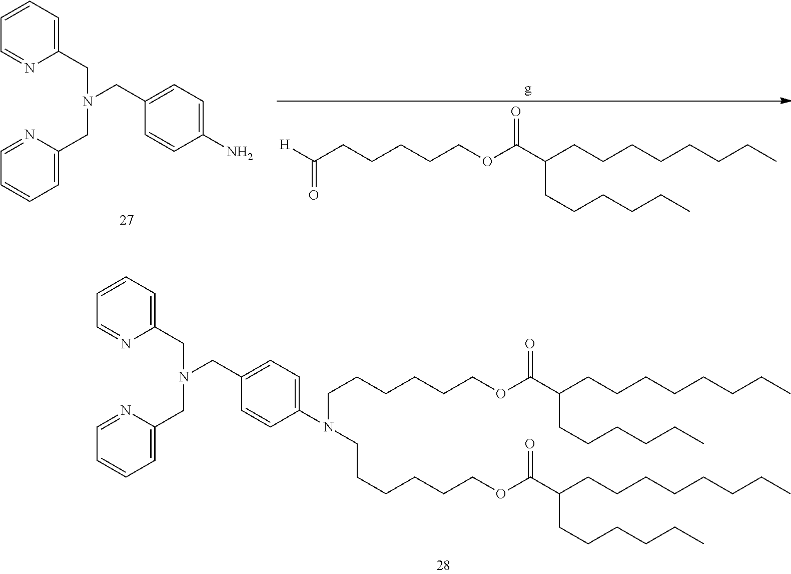 [SMILES: Nc1ccc(CN(Cc2ccccn2)Cc2ccccn2)cc1 ; CCCCCCCCC(CCCCCC)C(=O)OCCCCCC=O ; CCCCCCCCC(CCCCCC)C(=O)OCCCCCCN(CCCCCCOC(=O)C(CCCCCC)CCCCCCCC)c1ccc(CN(Cc2ccccn2)Cc2ccccn2)cc1]

Step g: Compound 27 (0.50 mmol), 6-oxohexyl 2-hexyldecanoate (1.00 mmol), acetic acid (0.80 mmol), and NaBH(OAc)₃ (3.00 mmol) were dissolved in 5 mL of DCM in the presence of nitrogen, followed by stirring at room temperature for 1 hour. After terminating the reaction by adding 2M aqueous NaOH solution, organic matter was extracted using DCM, and excess water was removed with MgSO₄. After removing the solvent through decompression distillation, the mixture was separated using basic alumina column chromatography (ethyl acetate:hexane=1:2) to obtain Compound 28 (62.6 mg, 7% yield) in light brown oil.

¹H NMR (400 MHz, CDCl₃): δ 8.50 (d, J=5.01 Hz, 2H), 7.64 (d, J=4.65 Hz, 4H), 7.33-7.20 (m, 2H), 7.12 (q, J=4.46 Hz, 2H), 6.57 (d, J=8.44 Hz, 2H), 4.05 (t, J=6.60 Hz, 4H), 3.88 (s, 4H), 3.67 (s, 2H), 3.21 (t, J=7.64 Hz, 4H), 2.42-2.19 (m, 2H), 1.76-1.46 (m, 12H), 1.38 (ddd, J=19.93, 9.23, 4.58 Hz, 12H), 1.24 (d, J=7.34 Hz, 40H), 0.84 (t, J=6.72 Hz, 12H).

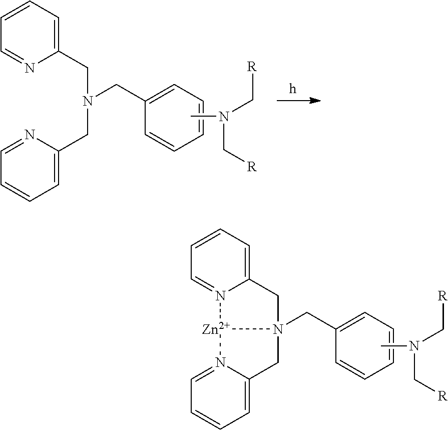

Step h: The lipid (0.1 mmol) synthesized in the presence of nitrogen and Zn²⁺ salts (0.1 mmol) were dissolved in 1.0 mL of MeCN and then stirred at room temperature for 3 hours, and the solvent was removed through decompression distillation.

Zn/DPA-03: (Compound with Chemical Formula 13)

¹H NMR (400 MHz, CDCl₃): δ 8.82 (d, J=5.38 Hz, 2H), 7.96 (t, J=7.58 Hz, 2H), 7.59-7.47 (m, 2H), 7.38 (d, J=8.56 Hz, 2H), 6.84-5.87 (m, 4H), 4.35 (d, J=15.41 Hz, 2H), 4.09-3.83 (m, 6H), 3.53 (s, 2H), 3.21 (t, J=7.52 Hz, 4H), 2.24 (tt, J=9.05, 5.38 Hz, 2H), 1.69-1.47 (m, 14H), 1.44-1.28 (m, 14H), 1.18 (s, 34H), 0.79 (td, J=6.91, 2.32 Hz, 14H).

Zn/DPA-04: (Compound with Chemical Formula 14)

¹H NMR (400 MHz, CDCl₃): δ 8.87 (d, J=5.26 Hz, 2H), 7.99 (t, J=7.76 Hz, 2H), 7.64-7.50 (m, 2H), 7.45 (d, J=7.83 Hz, 2H), 6.82 (d, J=78.13 Hz, 4H), 4.33 (d, J=15.53 Hz, 2H), 4.05 (s, 4H), 3.96 (d, J=15.53 Hz, 2H), 3.58 (s, 2H), 3.30 (s, 4H), 2.30 (tt, J=9.05, 5.32 Hz, 2H), 1.60 (dd, J=22.44, 6.30 Hz, 14H), 1.48-1.35 (m, 14H), 1.24 (s, 45H), 0.85 (t, J=5.81 Hz, 13H).

### <Example 2> Preparation of Lipid Nanoparticles

Lipid nanoparticles were prepared with 0.625 mg/mL CleanCap EGFP mRNA solution (50 mM sodium citrate buffer, 110 mM NaCl, pH=4.0) and lipid mixture solution (Ethanol) using laboratory mixers and emulsifiers (Namoassemblr Spark, Precision Nanosystems, Inc.) and provided by undergoing solvent conversion with normal saline or DPBS using a filter tube (UFC5010, Amicon) for centrifugation.

Specifically, lipid nanoparticles were prepared in the same content as Table 1 below.

TABLE 1

| | | | PEG-containing | | |
|---|---|---|---|---|---|
| | Zn/DPA lipid | Helper lipid | lipid | Size | |
| No. | (mol %) | (mol %) | (mol%) | (nm) | PDI |
| PAG75 | Zn/DPA-PA | Stearyl gallate | DMG-PEG | 104.7 | 0.23 |
| | (75) | (23) | (2) | | |
| PAT75 | Zn/DPA-PA | Trehalose | DMG-PEG | 120.7 | 0.23 |
| | (75) | dioleate | (2) | | |
| | | (23) | | | |
| PAG80 | Zn/DPA-PA | Stearyl gallate | DMG-PEG | 84.1 | 0.31 |
| | (80) | (18) | (2) | | |
| PAT80 | Zn/DPA-PA | Trehalose | DMG-PEG | 118.4 | 0.18 |
| | (80) | dioleate | (2) | | |
| | | (18) | | | |
| OAG75 | Zn/DPA-OA | Stearyl gallate | DMG-PEG | 121.5 | 0.22 |
| | (75) | (23) | (2) | | |
| LAG75 | Zn/DPA-LA | Stearyl gallate | DMG-PEG | 128.2 | 0.25 |
| | (75) | (23) | (2) | | |
| LAT75 | Zn/DPA-LA | Trehalose | DMG-PEG | 98.0 | 0.28 |
| | (75) | dioleate | (2) | | |
| | | (23) | | | |
| LNAG75 | Zn/DPA-LNA | Stearyl gallate | DMG-PEG | 127.1 | 0.23 |
| | (75) | (23) | (2) | | |
| LNAT75 | Zn/DPA-LNA | Trehalose | DMG-PEG | 74.8 | 0.22 |
| | (75) | dioleate | (2) | | |
| | | (23) | | | |
| LNAG80 | Zn/DPA-LNA | Stearyl gallate | DMG-PEG | 132.5 | 0.24 |
| | (80) | (18) | (2) | | |
| LNAT80 | Zn/DPA-LNA | Trehalose | DMG-PEG | 83.9 | 0.30 |
| | (80) | dioleate | (2) | | |
| | | (18) | | | |

### <Experimental Example 1> Identification of Nucleic Acid Binding of Amphiphilic Lipid Compounds

To identify the binding affinity of amphiphilic lipid compounds and nucleic acids, fluorescence intensity was observed in samples of dihydroxy-4-sulformhyl coumarin solution containing amphiphilic lipid compounds and RNA with various concentrations. Dihydroxy-4-sulformethyl coumarin emits fluorescence in an aqueous solution, and when combined with Zn/DPA, fluorescence is quenched. Since RNA and dihydroxy-4-sulformethyl coumarin competitively bind to Zn/DPA, the more RNA binds to the amphiphilic lipid compound, the higher the intensity of fluorescence as dihydroxy-4-sulformethyl coumarin fails to bind and falls off. Thus, the higher the binding of the RNA to the amphiphilic lipid compound, the stronger the fluorescence of the sample solution will be observed at the low RNA concentration.

The experimental process is as follows. Compounds with Chemical Formulas 3 to 6 that are amphiphilic lipid compounds were dissolved in RNase free water, and compounds with Chemical Formulas 9, 10, 13, and 14 were in dimethyl sulfoxide (DMSO) at a concentration of 4 mM respectively, followed by dilution 11 times with RNase free water. After preparing 500 ng/μL of RNA stock solution, serial dilution was performed 9 times with RNase free water to prepare RNA solutions with a total of 10 concentrations. A HEPES buffer (20 mM, pH 7.2) containing dihydroxy-4-sulformethyl coumarin (20 μM) was loaded into a black 96-well plate, and the prepared amphiphilic lipid compound solution (40 μM) and RNA solution (0 to 19 μg/100 μL) with various concentrations were added. The fluorescence intensity was observed using a Cytation 5 multiplate reader instrument (excitation wavelength 347 nm, emission wavelength 480 nm), and the fluorescence intensity according to RNA concentration was graphed and fitted with Hill equation.

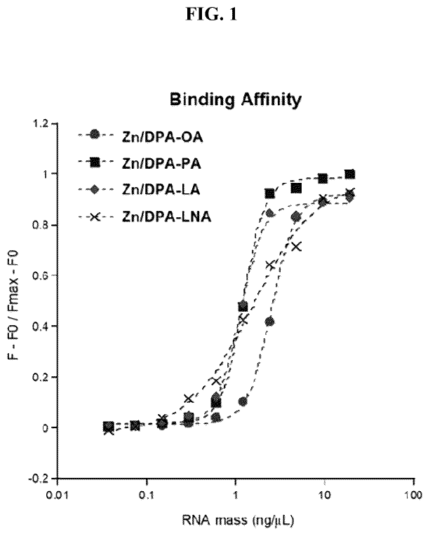

FIG. 1 shows a result of analyzing changes in fluorescence intensity of dihydroxy-4-sulformethyl coumarin according to RNA concentration for amphiphilic lipid compounds (Compounds with Chemical Formulas 3 to 6).

Referring to FIG. 1, the graph corresponding to the compound with Chemical Formula 4 is skewed to the right on the x-axis compared to the graph of the other compounds (Chemical Formulas 3, 5, and 6). This means that the binding affinity of the compounds with Chemical Formulas 3, 5, and 6 for RNA is higher than that of the compound with Chemical Formula 4. EC₅₀ (effect concentration of 50%), indicating the binding affinity, was 12, 25, 11, and 15 ng/μL for compounds with Chemical Formulas 3 to 6, respectively, with the highest binding affinity for compounds with Chemical Formulas 3 and 5 for RNA, and the lowest binding affinity for compounds with Chemical Formula 4. In addition, regarding a heel coefficient value, which represents the slope of the graph, the compound with Chemical Formula 6 has a relatively shallow slope (n=1.2) for RNA concentration. If the hill coefficient is greater than or equal to 1, it indicates that the higher the absolute value due to a cooperative reaction and the more RNA binds to the compound, the more likely it is that another RNA will bind to the compound. Therefore, the above results indicate that compounds with Chemical Formulas 3 to 5 bind sensitively by RNA concentrations, and the compound with Chemical Formula 6 binds insensitively by RNA concentrations.

The amphiphilic lipid compounds (compounds with Chemical Formulas 3 to 6) used in FIG. 1 had water-soluble linkers and were all soluble in water at a concentration of 40 mg/mL (about 0.05 M).

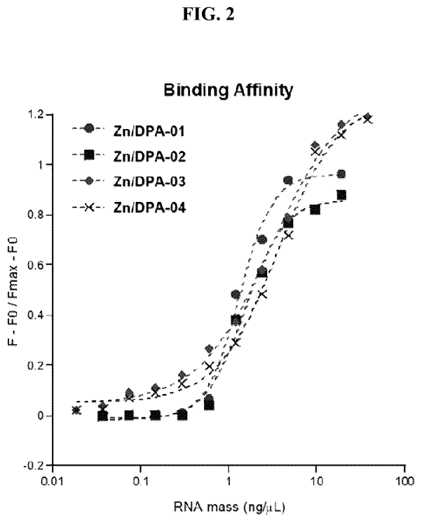

FIG. 2 shows a result of analyzing changes in fluorescence intensity of dihydroxy-4-sulformethyl coumarin according to RNA concentration for amphiphilic lipid compounds (compounds with Chemical Formulas 9, 10, 13, and 14).

Referring to FIG. 2, EC₅₀ (effect concentration of 50%), indicating the binding affinity, was 13, 15, 30, and 36 ng/μL for compounds with Chemical Formulas 9, 10, 13, and 14, respectively. Compounds with Chemical Formulas 9 and 10 including saturated hydrocarbon chains in the linker portion of amphiphilic lipid compounds have a higher binding affinity than compounds with Chemical Formulas 13 and 14 that include benzene/aromatic rings. In addition, since the compounds with Chemical Formulas 9 and 10 have a greater hill coefficient value than those with Chemical Formulas 13 and 14, it is noticed that the compounds with Chemical Formulas 9 and 10 bind more sensitively by RNA concentrations.

### <Experimental Example 2> Gel Electrophoresis of Amphiphilic Lipids

In order to determine a rate at which the amphiphilic lipid compound is able to fully capture nucleic acids, amphiphilic lipid compound-nucleic acid complexes were prepared by adjusting Zn/P, the ratio of phosphate residues of RNA to Zn of amphiphilic lipid compounds. Through gel electrophoresis, the amount of nucleic acids that failed to form a complex with amphiphilic lipid compounds was observed, and the appropriate Zn/P ratio was determined thereby.

The experimental process is as follows. Agarose powder was added to the water and heated in the microwave to completely dissolve the mixture. During the cooling process, 1× MOPS buffer, 7% formaldehyde, and 1× Midori green dye were added, mixed thoroughly, and then hardened in a gel tray to produce 1% agarose gel. The agarose gel prepared was soaked in an electrophoresis machine filled with 1× MOPS buffer. Based on 1 g of RNA, the amphiphilic lipid compound-nucleic acid complex was prepared to make the ratio (Zn/P) of phosphate residues of RNA to Zn of amphiphilic lipid compounds 4, 2, 1, 0.5, 0.25, and 0, respectively. 1× blue juice was mixed in the sample and loaded into each well of the agarose gel. Electrophoresis was proceeded at a voltage of 100 V for 10 minutes. Fluorescence was generated in the agarose gel with UV lamp to identify the amount of RNA that is not bound to the amphiphilic lipid compound.

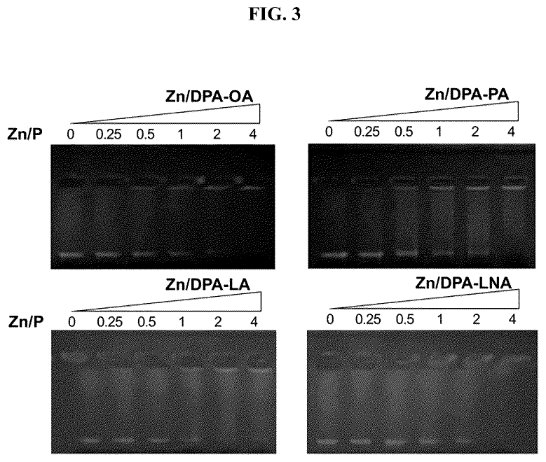

FIG. 3 shows results of analyzing gel chromatography of an amphiphilic lipid compound-nucleic acid complex according to Zn/P, which is a ratio of phosphate residues in RNA to Zn of amphiphilic lipid compounds (Compounds with Chemical Formulas 3 to 6).

Referring to FIG. 3, all compounds with Chemical Formulas 3 to 6 barely showed unbound RNA in gel chromatography when the Zn/P ratio is 4. In other words, if the Zn/P ratio is 4, it indicates that the amphiphilic lipid compound completely captured the nucleic acid.

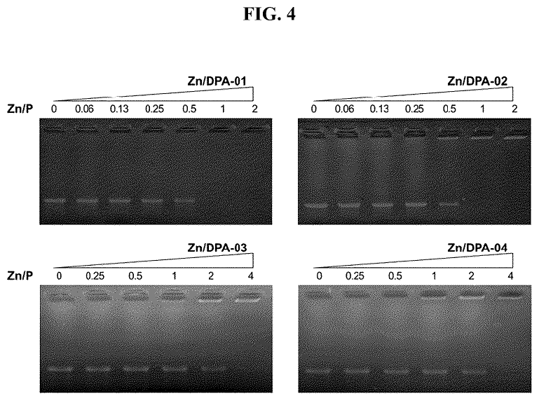

FIG. 4 shows results of analyzing gel chromatography of an amphiphilic lipid compound-nucleic acid complex according to Zn/P, which is a ratio of phosphate residues in RNA to Zn of amphiphilic lipid compounds (Compounds with Chemical Formulas 9, 10, 13, and 14).

Referring to FIG. 4, compounds with Chemical Formulas 9 and 10 barely showed RNA binding in electrophoresis when the Zn/P ratio is 1. On the other hand, compounds with Chemical Formulas 13 and 14 barely showed unbound RNA in gel chromatography when the Zn/P ratio is 4. At the Zn/P ratio, where unbound RNA was not observed, it indicates that the amphiphilic lipid compounds completely captured the nucleic acids. In addition, compounds with Chemical Formulas 9 and 10 had stronger binding to RNA than compounds with Chemical Formulas 13 and 14, indicating that the Chemical Formulas 13 and 14 showed similar binding as the Chemical Formulas 3 to 6.

### <Experimental Example 3> Measurement of Size and Polydispersity Index of Lipid Nanoparticles

In order to determine whether the lipid nanoparticles prepared using the amphiphilic lipid compounds (compounds with Chemical Formulas 3 to 6) exhibit the proper size and polydispersity index (PDI), the lipid nanoparticle solution presented in Table 1 was diluted 1,000 times with normal saline, followed by measurement.

The experimental process is as follows. A disposable sizing cell, DTS0012 cell (Malvern, USA), was used. 1 μL of each lipid nanoparticle solution in Table 1 or Table 2 was diluted with 999 μL of normal saline in DTS0012 cells and then covered up. Cells containing the diluted lipid nanoparticle solution were mounted by being oriented toward the Zetasizer Ultra red (Malvern, USA) instrument. Measurements were conducted using the ZS XPLORER program on a computer connected to the instrument. The size and polydispersity index measurement conditions were appropriately set in the program. After completing the measurement, the sample measurement status was checked in the Quality status window, and if the measurement status is blue, the lipid nanoparticle diluted solution was re-created in a new cell for measurement. If the measurement status is yellow/white, Z-Average (nm) and polydispersity index (PDI) data were secured from the size tab of the result window.

Table 1 shows results of the specific content, size, and polydispersity index of lipid nanoparticles, presenting results of the proper size and polydispersity index among lipid nanoparticles prepared using amphiphilic lipid compounds (compounds with Chemical Formulas 3 to 6). All were packed up except for the lipid nanoparticles exhibiting multiple sizes.

As shown in Table 1, of lipid nanoparticles using amphiphilic lipid compounds (compounds with Chemical Formulas 3 to 6), those prepared using 75 mol % or 80 mol % of compounds with Chemical Formulas 3 and 6 showed proper size and polydispersity index. On the other hand, compounds with Chemical Formulas 4 and 5 showed proper size and polydispersity index only in the preparation using 75 mol %. Compounds with Chemical Formulas 3, 5, and 6 showed proper size and polydispersity index in lipid nanoparticles prepared using stearyl gallate or trehalose dioleate as the helper lipid, while compounds with Chemical Formula 4 showed proper size and polydispersity index only in lipid nanoparticles prepared using stearyl gallate as the helper lipid.

As described above, since a specific part of the content of the present disclosure is described in detail, for those of ordinary skill in the art, it is clear that the specific description is only a preferred embodiment, and the scope of the present disclosure is not limited thereby. Thus, the substantial scope of the present disclosure will be defined by the appended claims and their equivalents.

What is claimed is:

1. A compound selected from a compound represented by the following Chemical Formula 1 or a pharmaceutically acceptable salt thereof: 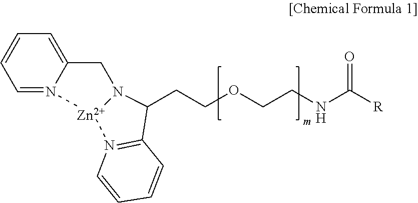
wherein, in the Chemical Formula 1,
R is a linear or branched, saturated or unsaturated hydrocarbon with 6 to 22 carbon atoms,
where the hydrocarbon comprises or does not comprise an ester, ether, amide, carbamate, carbonate, or disulfide bond, and
the ester, ether, amide, carbamate, carbonate, or disulfide bond is bonded or not bonded to a glycerol structure, and
m is an integer of 1 to 10.

2. The compound of claim 1, wherein the compound represented by Chemical Formula 1 is a compound represented by the following Chemical Formula 2: 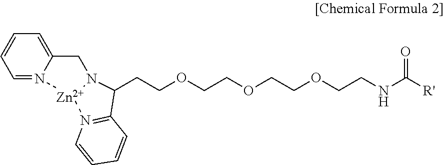
wherein, in the Chemical Formula 2,
R′ is a linear or branched, saturated or unsaturated hydrocarbon with 6 to 22 carbon atoms,
where the hydrocarbon comprises or does not comprise an ester, ether, amide, carbamate, carbonate, or disulfide bond.

3. The compound of claim 1, wherein the compound represented by Chemical Formula 1 comprises any one or more selected from the group consisting of compounds represented by the following Chemical Formula 3 to Chemical Formula 6:

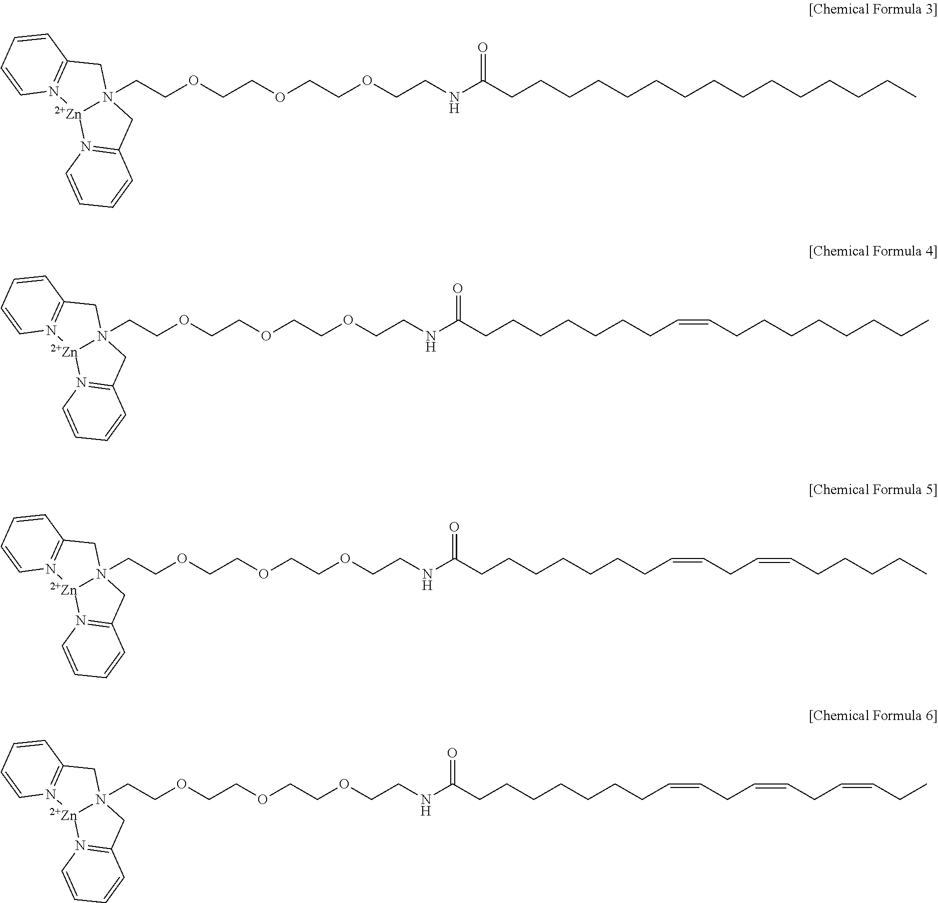

4. The compound of claim 1, wherein the compound is an amphiphilic lipid compound for nucleic acid binding.

5. A compound selected from a compound represented by the following Chemical Formula 7 or a pharmaceutically acceptable salt thereof: 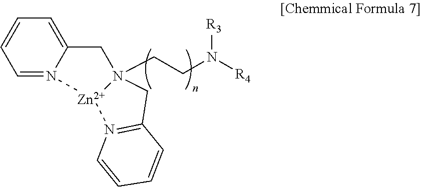
wherein, in the Chemical Formula 7,
R₃ and R₄ are the same or different and each independently linear or branched, saturated or unsaturated hydrocarbons with 6 to 22 carbon atoms,
where the hydrocarbon comprises or does not comprise an ester, ether, amide, carbamate, carbonate, or disulfide bond, and
the ester, ether, amide, carbamate, carbonate, or disulfide bond is bonded or not bonded to a glycerol structure, and
n is an integer of 0 to 10.

6. The compound of claim 5, wherein the compound represented by Chemical Formula 7 is a compound represented by the following Chemical Formula 8: 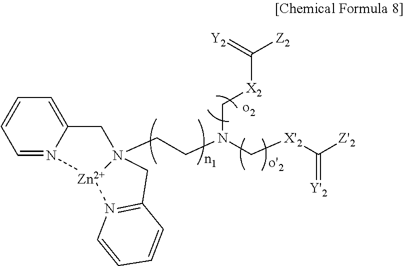
wherein, in the Chemical Formula 8,
X₂ and X′₂ are the same or different and each independently CH₂, NH, or O,
Y₂ and Y′₂ are the same or different and each independently O or S,
Z₂ and Z′₂ are the same or different and each independently linear or branched, saturated or unsaturated hydrocarbons with 6 to 22 carbon atoms,
n₁ is an integer of 0 to 5, and
o₂ and o′₂ are the same or different and each independently integers of 2 to 10.

7. The compound of claim 5, wherein the compound represented by Chemical Formula 7 comprises a compound represented by the following Chemical Formula 9 or Chemical Formula 10:

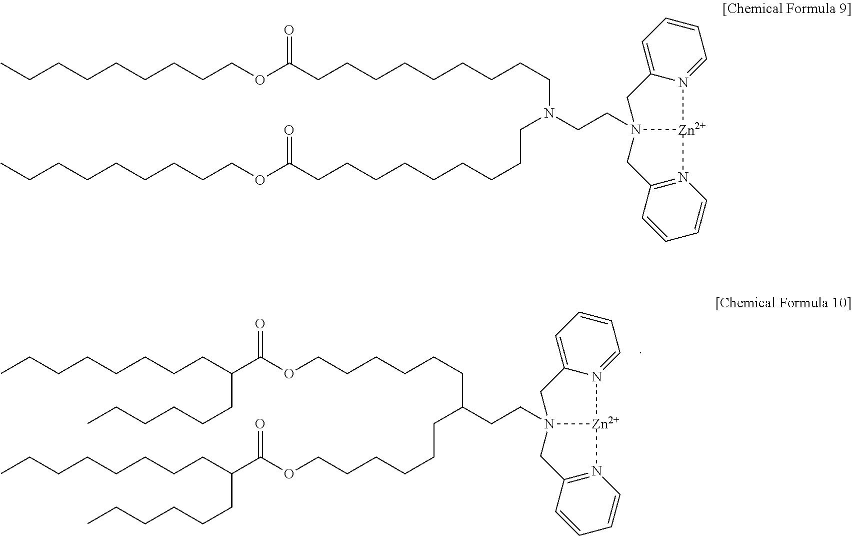

8. The compound of claim 5, wherein the compound is an amphiphilic lipid compound for nucleic acid binding.

9. A method of delivering a nucleic acid to a cell in a subject, comprising:
administering a lipid nanoparticle composition comprising the compound according to claim 1 and the nucleic acid to the subject.

10. The method of claim 9, wherein the composition further comprises one or more selected from the group consisting of a helper lipid and a PEGylated lipid.

11. The method of claim 10, wherein the helper lipid is selected from stearyl gallate or trehalose dioleate.

12. The method of claim 10, wherein the PEGylated lipid is selected from the group consisting of PEG-modified phosphatidylethanolamine, PEG-modified phosphatidic acid, PEG-modified ceramide, PEG-modified dialkylamine, PEG-modified diacylglycerol, PEG-modified dialkyl glycerol, and a mixture thereof.

13. The method of claim 10, wherein the composition comprises 50 to 80 mol % of the compound, 5 to 20 mol % of the helper lipid, and 1 to 5 mol % of the PEGylated lipid.

14. The method of claim 9, wherein the composition further comprises a therapeutic or prophylactic agent.

15. The method of claim 14, wherein the therapeutic or prophylactic agent is a vaccine or compound capable of inducing an immune response.

16. The method of claim 14, wherein the therapeutic or prophylactic agent is selected from the group consisting of small interfering RNA (siRNA), asymmetric interfering RNA (aiRNA), microRNA (miRNA), dicer-matrix RNA (dsRNA), small hairpin RNA (shRNA), messenger RNA (mRNA), and a mixture thereof.

17. A method of delivering a nucleic acid to a cell in a subject, comprising:
administering a lipid nanoparticle composition comprising the compound according to claim 5 and the nucleic acid to the subject.
